(19) United States
(12) Patent Application Publication
(10) Pub. No.: US 2021/0147761 A1
(43) Pub. Date: May 20, 2021

(54) THERMOLABILE PRO-FRAGRANCES OF FRAGRANCE KETONES

(71) Applicant: Henkel AG & Co. KGaA, Duesseldorf (DE)

(72) Inventors: Sascha Wilhelm SCHAEFER, Mettmann (DE); Silvia SAUF, Velbert (DE)

(21) Appl. No.: 16/756,881

(22) PCT Filed: Sep. 25, 2018
(86) PCT No.: PCT/EP2018/075899

§ 371 Date: Apr. 17, 2020

(30) Foreign Application Priority Data
Oct. 20, 2017 (DE) 10 2017 124 611.8

(51) Int. Cl. C11B 9/00; C11D 3/50; A61K 8/35; A61L 9/01

(52) U.S. Cl. CPC C11B 9/0015; C11B 9/0061; A61L 9/01; A61K 8/35; C11D 3/50

(57) ABSTRACT

Compositions comprising pro-fragrances of fragrance ketones may be suitable for perfuming laundry, washing and cleaning agents, since they respectively release the respective ketones during cleavage. Such pro-fragrances may include the formula (I):

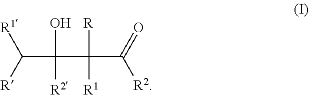

### CROSS-REFERENCE TO RELATED APPLICATIONS

The present application is a national stage entry according to 35 U.S.C. § 371 of PCT application No.: PCT/EP2018/075899 filed on Sep. 25, 2018; which claims priority to German Patent Application Serial No.: 10 2017 124 611.8, which was filed on Oct. 20, 2017; which are incorporated herein by reference in its entirety and for all purposes.

### TECHNICAL FIELD

Pro-fragrances of fragrance ketones are suitable, for example, for the fragrancing of laundry, since they release the respective ketones during cleavage.

### BACKGROUND

The controlled release of fragrances in the field of washing and cleaning agents in order to intensively and long-lastingly fragrance the product as well as the washing and cleaning solution and the articles treated using said agents is known in the prior art.

A basic problem associated with the use of fragrances is that they are more or less highly volatile compounds, although a long-lasting fragrance effect is desired. In particular in the case of fragrances that produce the fresh and light notes of the perfume and evaporate particularly quickly due to their relatively high vapor pressure, it is difficult to achieve the desired long-lasting impression of fragrance.

Pro-fragrance molecules, which are, for example, hydrolytically labile or photolabile, are known in the prior art, and represent one option for the delayed release of fragrances. The effect of environmental factors causes splitting of a covalent bond in the pro-fragrance molecule, thereby releasing a fragrance.

### SUMMARY

The inventors have now surprisingly found that such compounds can be prepared by dimerizing fragrance ketones, the thermally catalyzed cleavage of which during or after use produces the monomers again.

In a first aspect are pro-fragrances of formula (I)

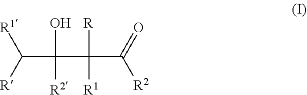

wherein
R, R′, R¹, R^{1′}, R² and R^{2′} are independently selected from H, straight-chain or branched, saturated or unsaturated, substituted or unsubstituted hydrocarbon groups having 1 to 20 carbon atoms and optionally up to 6 heteroatoms, such as linear or branched alkyl, alkenyl or alkynyl having up to 20, such as up to 12, carbon atoms, substituted or unsubstituted, linear or branched heteroalkyl, heteroalkenyl or heteroalkynyl having up to 20, such as up to 12, carbon atoms, and 1 to 6, such as 1 to 4, heteroatoms selected from O, S and N, substituted or unsubstituted aryl having up to 20, such as up to 12, carbon atoms, substituted or unsubstituted heteroaryl having up to 20, such as up to 12, carbon atoms, and 1 to 6, such as 1 to 4, heteroatoms selected from O, S and N, cycloalkyl or cycloalkenyl having up to 20, such as up to 12, carbon atoms, and heterocycloalkyl or heterocycloalkenyl having up to 20, such as up to 12, carbon atoms, and 1 to 6, such as 1 to 4, heteroatoms selected from O, S and N, or R² and R¹ or R and R² or R^{2′} and R^{1′} or R^{2′} and R′ may combine to form a cyclic group selected from substituted or unsubstituted aryl having up to 20, such as up to 12, carbon atoms, substituted or unsubstituted heteroaryl having up to 20, such as up to 12, carbon atoms, and 1 to 6, such as 1 to 4, heteroatoms selected from O, S and N, substituted or unsubstituted cycloalkyl or cycloalkenyl having up to 20, such as up to 12, carbon atoms, and substituted or unsubstituted heterocycloalkyl or heterocycloalkenyl having up to 20, such as up to 12, carbon atoms, and 1 to 6, such as 1 to 4, heteroatoms selected from O, S and N, with the proviso that at least one of R, R¹ and R² and at least one of R′, R^{1′} and R^{2′} is not H and the group —(CRR¹)—C(O)R² is derived from a fragrance ketone of formula R²—C(O)—CHRR¹ and the group —COHR^{2′}—CR′R^{1′} is derived from a fragrance ketone of formula R^{2′}—C(O)—CHR′R^{1′}.

The compounds mentioned can be prepared by means of the synthesis routes described in the examples.

In a further aspect, the use of compounds of formula (I) are described herein as a fragrance in liquid or solid washing and cleaning agents or in cosmetic agents, in particular those for skin or hair treatment, optionally together with other fragrances, in air care products or in insect repellents, or to prolong the fragrance effect of other fragrances.

Yet another aspect is directed to agents containing the pro-fragrances described herein, in particular washing or cleaning agents, cosmetic agents, air care products or insect repellents.

Lastly, a method for the long-lasting fragrancing of surfaces in which a compound according to formula (I) as described herein is applied to the surface to be fragranced, and this surface is subsequently exposed to conditions which lead to the fragrance being released. The surface to be fragranced may be, for example, (textile) laundry.

### DETAILED DESCRIPTION

“At least one” as used herein refers to 1 or more, for example 2, 3, 4, 5, 6, 7, 8, 9 or more. In connection with components of the compound described herein, this statement refers not to the absolute amount of molecules, but rather to the type of component. “At least one compound of formula X” therefore means, for example, one or more different compounds of formula X, i.e. one or more different types of compounds of formula X. Together with the stated amounts, the stated amounts refer to the total amount of the correspondingly designated type of component, as already defined above.

All amounts stated in connection with the agents described herein refer to wt. %, in each case based on the total weight of the agent, unless indicated otherwise. Moreover, stated amounts of this kind that relate to at least one component always relate to the total amount of this type of component contained in the agent, unless explicitly indicated otherwise. This means that stated amounts of this kind, for example in connection with “at least one fragrance,” refer to the total amount of fragrance contained in the agent.

As described herein, the term “fragrance ketones” is understood to mean fragrances which have a keto group, regardless of how the molecule is further structured. It is necessary in various embodiments that the corresponding ketones are deprotonatable in the alpha position, i.e., at least one H is bonded to the alpha C atom. Such ketones that are deprotonatable in the alpha-position are therefore the fragrance ketones that form the pro-fragrances. The terms “odorant” and “fragrance” are used interchangeably herein and refer in particular to substances that have a scent that is perceived to be pleasant by humans. In various embodiments, fragrances are those substances that are sufficiently volatile to be perceived as odorous by humans by binding to the olfactory receptor, and the odor of which is perceived as pleasant. The fragrances or odorants are in particular those which are suitable for use in cosmetic, cleaning agent or washing agent compositions. Generally, the fragrance or odorant is liquid at ambient temperatures.

Suitable fragrance ketones include, but are not limited to, 2-undecanone (methyl nonyl ketone), methyl beta naphthyl ketone, musk indanone (1,2,3,5,6,7-hexahydro-1,1,2,3,3-pentamethyl-4H-inden-4-one), tonalide (6-acetyl-1,1,2,4,4,7-hexamethyltetralin), alpha-damascone, beta-damascone, delta-damascone, iso-damascone, damascenone, methyldihydrojasmonate, menthone, carvone, camphor, koavone (3,4,5,6,6-pentamethylhept-3-en-2-one), fenchone, alpha-ionone, beta-ionone, gamma-methyl-ionone, fleuramone (2-heptylcyclopentanone), dihydrojasmone, cis-jasmone, 1-(1,2,3,4,5,6,7,8-octahydro-2,3,8,8-tetramethyl-2-naphthalenyl)-ethan-1-one and isomers thereof, methyl cedrenyl ketone, acetophenone, methyl acetophenone, para-methoxy acetophenone, methyl beta-naphthyl ketone, benzyl acetone, benzophenone, para-hydroxyphenyl butanone, celery ketone (3-methyl-5-propyl-2-cyclohexenone), 6-isopropyldecahydro-2-naphtone, dimethyloctenone, Frescomenthe (2-butan-2-yl-cyclohexan-1-one), 4-(1-ethoxyvinyl)-3,3,5,5-tetramethylcyclohexanone, methyl heptenone, 2-(2-(4-methyl-3-cyclohexen-1-yl)propyl)cyclopentanone, 1-(p-menthen-6(2)yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)-indanone, 4-damascol, dulcinyl (4-(1,3-benzodioxol-5-yl)butan-2-one), hexalone (1-(2,6,6-trimethyl-2-cyclohexen-1-yl)-1,6-heptadien-3-one), Isocyclemone E (2-acetonaphthone-1,2,3,4,5,6,7,8-octahydro-2,3,8,8-tetramethyl), methyl nonyl ketone, methyl cyclocitrone, methyl lavender ketone, orivone (4-tert-amylcyclohexanone), 4-tert-butylcyclohexanone, delphone (2-pentyl-cyclopentanone), muscone (CAS 541-91-3), neobutenone (1-(5,5-dimethyl-1-cyclohexenyl)pent-4-en-1-one), plicatone (CAS 41724-19-0), veloutone (2,2,5-trimethyl-5-pentylcyclopentan-1-one), 2,4,4,7-tetramethyl-oct-6-en-3-one and tetrameran (6,10-dimethylundecen-2-one), and mixtures thereof.

In addition, as fragrance ketones basically all the usual fragrance ketones can be used which are used, in particular, to bring about a pleasant olfactory sensation for humans. Such fragrance ketones are known to a person skilled in the art and are also described in the patent literature, for example in US 2003/0158079 A1, paragraphs [0154] and [0155]. For further suitable odorants, reference should be made to Steffen Arctander, Aroma Chemicals Volume 1 and Volume 2 (published in 1960 and 1969, reissue 2000; ISBN: 0-931710-37-5 and 0-931710-38-3).

In various embodiments, the pro-fragrances are those resulting from fragrance ketones, in particular those mentioned above. In various embodiments, the fragrance ketones are those in which neither the alpha carbon atom nor the beta carbon atom (in each case relative to the oxygen atom) is part of a cyclic group.

In various embodiments, the pro-fragrances are dimers of the same fragrance ketone, i.e., the two fragrance ketones that form the compound are structurally identical.

In various embodiments, R²/R^{2′} is a straight-chain or branched, saturated or unsaturated, substituted or unsubstituted hydrocarbon group having 1 to 20 carbon atoms and optionally up to 6 heteroatoms, such as a linear or branched alkyl, alkenyl or alkynyl group having up to 20, such as up to 12, carbon atoms, such as methyl, ethyl, propyl, butyl, pentyl, hexyl, heptyl, octyl, nonyl or decyl.

In various embodiments, R¹/R^{1′} or R/R′ is H and the other group is a straight-chain or branched, saturated or unsaturated, substituted or unsubstituted hydrocarbon group having 1 to 20 carbon atoms and optionally up to 6 heteroatoms, such as a linear or branched alkyl, alkenyl or alkynyl group having up to 20, such as up to 12, carbon atoms. In various embodiments, R¹R^{1′} and R/R′ may also be H.

In the following, further embodiments of R, R¹ and R² are described by way of example, it being intended that the same embodiments are equally applicable to R′, R^{1′} and R^{2′}, either alternatively or in combination. In particular, each embodiment defining R, R¹ and R² may equally define R′, R^{1′} and R^{2′}.

If R² and R¹ are combined to form a cyclic group, this is selected from substituted or unsubstituted aryl having up to 20, such as up to 12, carbon atoms, substituted or unsubstituted heteroaryl having up to 20, such as up to 12, carbon atoms, and 1 to 6, such as 1 to 4, heteroatoms selected from O, S and N, substituted or unsubstituted cycloalkyl or cycloalkenyl having up to 20, such as up to 12, carbon atoms, and substituted or unsubstituted heterocycloalkyl or heterocycloalkenyl having up to 20, such as up to 12, carbon atoms, and 1 to 6, such as 1 to 4, heteroatoms selected from O, S and N, particularly such as cycloalkyl or cycloalkenyl as defined above.

Generally, in various embodiments, R, R¹ and R² are selected to form, together with the two carbon atoms to which they are bonded, an organic group having at least 6 carbon atoms.

In various embodiments, R¹ and Rare Hand R² is a linear, optionally substituted, alkyl group having up to 12 carbon atoms. When substituted, the substituent is a cyclic group, for example an aryl or heteroaryl ring, a cycloalkyl or heterocycloalkyl group, such as having 5-6 carbon atoms, e.g. an aryl group.

“Substituted” as used herein means that one or more hydrogen atoms in the corresponding group are replaced by another group, such as selected from hydroxyl, carboxyl, amino, halogen, (hetero)alkyl, (hetero)alkenyl, (hetero)alkynyl, (hetero)aryl, (hetero)cycloalkyl, and (hetero)cycloalkenyl, with the proviso that a given group cannot be substituted with a group of the same kind (i.e., for example, alkyl with alkyl). Substituted groups are alkylaryl or arylalkyl groups.

“Groups” of the above-mentioned fragrance ketones are the corresponding fragrance ketones in which a hydrogen atom is replaced by the group —COHR^{2′}—C^{R′}R^{1′} at the beta-carbon atom or a fragrance ketone in which the carbonyl group is replaced by a group of formula —COH—CRR¹—C(O)R².

The pro-fragrances are characterized in that they release the fragrance ketones via thermolysis over a sustained period of time. They can also be used in aqueous media or in processes for producing granules, without suffering excessive loss of activity. In this way, liquid washing and cleaning agents such as liquid washing agents, fabric softeners, hand dishwashing agents, cleaning agents for hard surfaces, floor wipes, etc. are also conceivable, as are solid washing and cleaning agents, for example textile washing agent granules, automatic dishwasher detergents or cleaning and scouring agents. Likewise, the pro-fragrances can be used in cosmetic agents for skin and hair treatment. This also involves both liquid agents, such as shower gels, deodorants and hair shampoo, as well as solid agents, such as bars of soap.

Due to the excellent suitability of the compounds for use in washing agents and cleaning agents, the use of pro-fragrances as described herein relates to fragrance in liquid or solid washing and cleaning agents and in cosmetic agents, in particular those for skin and hair treatment, but also air care agents and insect repellents.

The pro-fragrances can be introduced in varying amounts depending on the nature and intended use of the agents to be fragranced. Usually, the pro-fragrances are used in washing and cleaning agents in amounts of from 0.001 to 5 wt. %, such as from 0.01 to 2 wt. %, in each case based on the agent concerned. The agents may include a pro-fragrance or a plurality of different pro-fragrances as described herein, with the above stated amounts referring to the total amount of all pro-fragrances. In insect repellents, the amounts used can be significantly higher, for example concentrations of from 0.001 to 100 wt. %, such as 1 to 50 wt. %, in each case based on the agent, can be used here.

The pro-fragrances can be used as the sole fragrance, but it is also possible to use fragrance mixtures which consist only in part of the pro-fragrances. Thus, in particular fragrance mixtures can be used which contain 1 to 50 wt. %, such as 5 to 40 and in particular at most 30 wt. % of pro-fragrances based on the fragrance mixture. In other embodiments, in which in particular the delayed fragrance effect of the pro-fragrances is to be used, in the use advantageously at least 30 wt. %, such as at least 40 wt. % and in particular at least 50 wt. % of the total perfume contained in the agent are introduced into the agents via the pro-fragrances, while the remaining 70 wt. %, such as 60 wt. % and in particular 50 wt. % of the total perfume contained in the agent are sprayed on in a conventional manner or otherwise introduced into the agents in another manner. The use can therefore advantageously be characterized in that the pro-fragrances are used together with other fragrances.

By dividing the total perfume content of the agents into perfume which is contained in the pro-fragrances and perfume which has been incorporated conventionally, a variety of product characteristics can be achieved, which are only possible by means of the use. Thus, for example, it is conceivable and possible to divide the total perfume content of the agent into two portions x and y, wherein proportion x consists of adherent, i.e. less-volatile, perfume oils, and proportion y consists of more-volatile perfume oils.

Washing or cleaning agents can now be prepared, for example, in which the proportion of perfume which is introduced via the pro-fragrances into the agents, is mainly composed of adherent odorants. In this way, adherent odorants, which are intended to fragrance the treated articles, in particular textiles, are “held” in the product and thereby exert their effect mainly on the treated laundry. In contrast, the more-volatile odorants contribute to a more intensive fragrancing of the agents themselves. In this way, it is also possible to prepare washing and cleaning agents which as agents have an odor which differs from the odor of the treated articles. There are hardly any limits to the creativity of perfumers, as the choice of fragrances and the choice of method of incorporating said fragrance into the agents give almost limitless possibilities for fragrancing the agents and, by means of the agents, objects treated by said agents.

Of course, the principle described above can also be reversed by incorporating the more-volatile fragrances into the pro-fragrances and spraying or otherwise incorporating the less-volatile, adherent fragrances onto the agents. In this way, the loss of the more-volatile fragrances from the packaging during storage and transport is minimized while the fragrance characteristic of the agents is determined by the more adherent perfumes. The use of the more-volatile fragrances in the form of the pro-fragrances described herein may be used in various embodiments.

The only limitation of this approach is that the fragrances which are to be introduced via the pro-fragrances come from the group of fragrance ketones. The fragrances incorporated into the agents in a conventional manner are not subject to any restrictions. It is possible, for example, to use individual fragrance compounds, such as the synthetic products of the ester, ether, aldehyde, ketone, alcohol, and hydrocarbon types, as perfume oils or fragrances. Fragrance compounds of the ester type are, for example, benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, floramate, melusate, and jasmacyclate. The ethers include, for example, benzyl ethyl ether and ambroxan; the aldehydes include, for example, the linear alkanals having 8 to 18 carbon atoms, citral, citronellal, citronellyloxyacetaldehyde, cyclamenaldehyde, Lilial, and bourgeonal; the ketones include, for example, the ionones, α-isomethylionone, and methyl cedryl ketone; the alcohols include, anethole, citronellol, eugenol, geraniol, linalool, phenylethyl alcohol, and terpineol; and the hydrocarbons include principally the terpenes such as limonene and pinene. In a non-limiting embodiment, however, mixtures of different odorants are used, which together produce an appealing fragrance note. Suitable aldehydes are disclosed in the sources described above in the context of the suitable ketones.

Perfume oils of this type may also contain natural odorant mixtures as can be obtained from plant sources such as pine, citrus, jasmine, patchouli, rose or ylang-ylang oil. Likewise suitable are clary sage oil, chamomile oil, clove oil, melissa oil, mint oil, cinnamon leaf oil, lime blossom oil, juniper berry oil, vetiver oil, olibanum oil, galbanum oil, and labdanum oil, as well as orange blossom oil, neroli oil, orange peel oil, and sandalwood oil.

The general description of perfumes that can be used (see above) generally represents the different substance classes of odorants. In order to be perceptible, an odorant must be volatile, the molar mass also playing an important role in addition to the nature of the functional groups and the structure of the chemical compound. Therefore, most odorants have molar masses of up to approximately 200 daltons, whereas molar masses of 300 daltons and above are something of an exception. Due to the differing volatility of odorants, the odor of a perfume or fragrance composed of multiple odorants varies over the course of vaporization, the odor impressions being divided into “top note”, “middle note or body” and “end note or dry out.” Because the perception of an odor also depends to a large extent on the odor intensity, the top note of a perfume or fragrance is not made up only of highly volatile compounds, while the end note comprises for the most part less-volatile, i.e., adherent odorants. When composing perfumes, more-volatile fragrances can be bound, for example, to specific fixatives, thereby preventing them from evaporating too quickly. The above-described embodiment in which the more-volatile fragrances are present in the pro-fragrances is one such odorant-fixing method. For the following subdivision of odorants into “less-volatile” and “adherent” fragrances, there is therefore no mention of the odor impression, and, moreover, as to whether the corresponding odorant is perceived as a top or middle note.

Adherent odorants that can be used are, for example, essential oils such as angelica root oil, anise oil, arnica blossom oil, basil oil, bay oil, champaca blossom oil, abies alba oil, abies alba cone oil, elemi oil, eucalyptus oil, fennel oil, spruce needle oil, galbanum oil, geranium oil, ginger grass oil, guaiac wood oil, gurjun balsam oil, helichrysum oil, ho oil, ginger oil, iris oil, cajeput oil, calamus oil, chamomile oil, camphor oil, cananga oil, cardamom oil, cassia oil, pine needle oil, copaiba balsam oil, coriander oil, spearmint oil, caraway oil, cumin oil, lavender oil, lemon grass oil, lime oil, mandarin oil, melissa oil, musk seed oil, myrrh oil, clove oil, neroli oil, niaouli oil, olibanum oil, oregano oil, palmarosa oil, patchouli oil, balsam Peru oil, petitgrain oil, pepper oil, peppermint oil, pimento oil, pine oil, rose oil, rosemary oil, sandalwood oil, celery oil, spike oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil, and cypress oil. However, higher-boiling and solid odorants of natural or synthetic origin can also be used as adherent odorants or odorant mixtures, i.e. fragrances. These compounds include the compounds indicated in the following and mixtures thereof: Ambrettolide, Ambroxan, α-amylcinnamaldehyde, anethole, anisaldehyde, anise alcohol, anisole, anthranilic acid methyl ester, acetophenone, benzylacetone, benzaldehyde, benzoic acid ethyl ester, benzophenone, benzyl alcohol, benzyl acetate, benzyl benzoate, benzyl formate, benzyl valerianate, borneol, bornyl acetate, Boisambrene forte, α-bromostyrene, n-decyl aldehyde, n-dodecyl aldehyde, eugenol, eugenol methyl ether, eucalyptol, farnesol, fenchone, fenchyl acetate, geranyl acetate, geranyl formate, heliotropin, heptyne carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrole, jasmone, camphor, carvacrol, carvone, p-cresol methyl ether, coumarin, p-methoxyacetophenone, methyl n-amyl ketone, methylanthranilic acid methyl ester, p-methylacetophenone, methyl chavicol, p-methylquinoline, methyl-β-naphthyl ketone, methyl n-nonyl acetaldehyde, methyl n-nonyl ketone, muscone, β-naphthol ethyl ether, β-naphthol methyl ether, nerol, n-nonyl aldehyde, nonyl alcohol, n-octylaldehyde, p-oxyacetophenone, pentadecanolide, β-phenethyl alcohol, phenylacetaldehyde dimethyl acetal, phenylacetic acid, pulegone, safrole, salicylic acid isoamyl ester, salicylic acid methyl ester, salicylic acid hexyl ester, salicylic acid cyclohexyl ester, santalol, sandelice, skatole, terpineol, thymene, thymol, troenan, γ-undecalactone, vanillin, veratraldehyde, cinnamaldehyde, cinnamyl alcohol, cinnamic acid, cinnamic acid ethyl ester, and cinnamic acid benzyl ester.

More-volatile odorants include in particular lower-boiling odorants of natural or synthetic origin, which may be used alone or in mixtures. Examples of more-volatile odorants are diphenyloxide, limonene, linalool, linalyl acetate and propionate, melusate, menthol, menthone, methyl-n-heptenone, pinene, phenylacetaldehyde, terpinyl acetate, citral, and citronellal.

In addition to the described fragrances, the agents, for example washing and cleaning agents can, of course, contain customary ingredients of such agents. In this regard, in the case of washing and cleaning agents, primarily surfactants, builder substances, bleaching agents, enzymes, and other active substances should be mentioned. The essential ingredients of washing and cleaning agents include in particular surfactants.

Depending on the intended purpose of the agents, the surfactant content will be selected higher or lower. The surfactant content of washing agents can typically be, for example, between 10 and 40 wt. %, such as between 12.5 and 30 wt. %, and in particular between 15 and 25 wt. %, while cleaning agents for automatic dishwashing may contain between 0.1 and 10 wt. %, such as between 0.5 and 7.5 wt. %, and in particular between 1 and 5 wt. %, surfactants.

These surface-active ingredients come from the group of anionic, non-ionic, zwitterionic or cationic surfactants, anionic and non-ionic surfactants being used for economic reasons and due to the performance spectrum thereof during washing and cleaning.

Anionic surfactants that are used are those of the sulfonate and sulfate types, for example. Surfactants of the sulfonate type that can be used are C₉₋₁₃ alkylbenzene sulfonates, olefin sulfonates, i.e. mixtures of alkene and hydroxyalkane sulfonates, and disulfonates, as obtained, for example, from C₁₂₋₁₈ monoolefins having a terminal or internal double bond by way of sulfonation with gaseous sulfur trioxide and subsequent alkaline or acid hydrolysis of the sulfonation products. Alkane sulfonates obtained from C₁₂₋₁₈ alkanes, for example by way of sulfochlorination or sulfoxidation with subsequent hydrolysis or neutralization, are also suitable. Likewise, the esters of α-sulfofatty acids (ester sulfonates) are suitable, for example the α-sulfonated methyl esters of hydrogenated coconut fatty acids, palm kernel fatty acids or tallow fatty acids.

Sulfated fatty acid glycerol esters are further suitable anionic surfactants. Fatty acid glycerol esters shall be understood to mean the monoesters, diesters and triesters and mixtures thereof, as they are obtained during preparation by way of the esterification of a monoglycerol with 1 to 3 moles fatty acid or during the transesterification of triglycerides with 0.3 to 2 moles glycerol. Non-limiting sulfated fatty acid glycerol esters are the sulfation products of saturated fatty acids having 6 to 22 carbon atoms, for example of caproic acid, caprylic acid, capric acid, myristic acid, lauric acid, palmitic acid, stearic acid or behenic acid.

The alkali salts and in particular the sodium salts of the sulfuric acid half-esters of C₁₂-C₁₈ fatty alcohols, for example from coconut fatty alcohol, tallow fatty alcohol, lauryl alcohol, myristyl alcohol, cetyl alcohol or stearyl alcohol, or of C₁₀-C₂₀ oxo alcohols and the half-esters of secondary alcohols having these chain lengths are used as alk(en)yl sulfates. Alk(en)yl sulfates of the mentioned chain length that contain a synthetic straight-chain alkyl group prepared on a petrochemical basis and have a degradation behavior similar to that of the adequate compounds based on fat chemical raw materials are also used. From a washing perspective, the C₁₂-C₁₆ alkyl sulfates, C₁₂-C₁₅ alkyl sulfates and C₁₄-C₁₅ alkyl sulfates are used.

The sulfuric acid monoesters of straight-chain or branched C₇₋₂₁ alcohols ethoxylated with 1 to 6 mol of ethylene oxide, such as 2-methyl-branched C₉₋₁₁ alcohols having, on average, 3.5 mol ethylene oxide (EO) or C₁₂₋₁₈ fatty alcohols having 1 to 4 EO, are also suitable. Due to the high foaming behavior, they are used only in relatively small amounts in cleaning agents, for example in amounts of from 1 to 5 wt. %.

Further suitable anionic surfactants are also the salts of alkyl sulfosuccinic acid, which are also referred to as sulfosuccinates or as sulfosuccinic acid esters and represent monoesters and/or diesters of sulfosuccinic acid with alcohols, such as fatty alcohols, and in particular ethoxylated fatty alcohols. Non-limiting sulfosuccinates contain C₈₋₁₈ fatty alcohol groups or mixtures of these. In particular, sulfosuccinates contain a fatty alcohol group that is derived from ethoxylated fatty alcohols, which taken alone represent non-ionic surfactants (for description see below). Among these, in turn, sulfosuccinates including fatty alcohol groups that derive from ethoxylated fatty alcohols exhibiting a restricted distribution of homologs are used. Likewise, it is also possible to use alk(en)yl succinic acid having 8 to 18 carbon atoms in the alk(en)yl chain, or the salts thereof.

Further anionic surfactants that can also be used are in particular soaps. Saturated fatty acid soaps are suitable, such as the salts of lauric acid, myristic acid, palmitic acid, stearic acid, hydrogenated erucic acid and behenic acid, and in particular soap mixtures derived from natural fatty acids, such as coconut fatty acids, palm kernel fatty acids or tallow fatty acids.

The anionic surfactants, including the soaps, can be present in the form of the sodium, potassium or ammonium salts thereof, or as soluble salts of organic bases, such as monoethanolamine, diethanolamine or triethanolamine. The anionic surfactants are present in the form of the sodium, potassium or magnesium salts thereof, and in particular in the form of the sodium salts.

There are no general conditions that must be adhered to that would stand in the way of having a degree of freedom in terms of formulation when selecting the anionic surfactants. Non-limiting agents, however, have a soap content that exceeds 0.2 wt. %, based on the total weight of the washing and cleaning agent prepared in step d). Alkylbenzene sulfonates and fatty alcohol sulfates as anionic surfactants are used, such as shaped washing agent bodies containing 2 to 20 wt. %, such as 2.5 to 15 wt. %, or 5 to 10 wt. %, fatty alcohol sulfate(s), in each case based on the weight of the agents.

Non-ionic surfactants that are used are alkoxylated, advantageously ethoxylated, in particular primary alcohols having 8 to 18 C atoms and, on average, 1 to 12 mols of ethylene oxide (EO) per mol of alcohol, in which the alcohol group can be linear or methyl-branched in the 2 position, or can contain linear and methyl-branched groups in admixture, as are usually present in oxo alcohol groups. However, alcohol ethoxylates having linear groups of alcohols of native origin having 12 to 18 C atoms, for example of coconut, palm, tallow fatty or oleyl alcohol, and an average of 2 to 8 EO per mol of alcohol, are particularly possible. Examples of ethoxylated alcohols include C₁₂₋₁₄ alcohols having 3 EO or 4 EO, C₉₋₁₁ alcohol having 7 EO, C₁₃₋₁₅ alcohols having 3 EO, 5 EO, 7 EO or 8 EO, C₁₂₋₁₈ alcohols having 3 EO, 5 EO or 7 EO, and mixtures thereof, such as mixtures of C₁₂₋₁₄ alcohol having 3 EO and C₁₂₋₁₈ alcohol having 5 EO. The degrees of ethoxylation indicated represent statistical averages that can correspond to an integer or a fractional number for a specific product. Non-limiting alcohol ethoxylates have a narrowed homolog distribution (narrow range ethoxylates, NRE). In addition to these non-ionic surfactants, fatty alcohols having more than 12 EO can also be used. Examples of these are tallow fatty alcohols having 14 EO, 25 EO, 30 EO, or 40 EO.

Another class of used non-ionic surfactants, which are used either as the only non-ionic surfactant or in combination with other non-ionic surfactants, are alkoxylated, ethoxylated or ethoxylated and propoxylated fatty acid alkyl esters, having 1 to 4 carbon atoms in the alkyl chain, in particular fatty acid methyl esters, as they are described in the Japanese patent application JP 58/217598, for example, or prepared according to the method described in the international patent application WO-A-90/13533.

Another class of non-ionic surfactants that can advantageously be used is the alkyl polyglycosides (APG). Alkyl polyglycosides that can be used satisfy the general formula RO(G)_{z}, in which R represents a linear or branched, in particular methyl-branched at the 2-position, saturated or unsaturated aliphatic group having 8 to 22, such as 12 to 18, C atoms, and G is the symbol that represents a glycose unit having 5 or 6 C atoms, e.g. glucose. The degree of glycosidation z is between 1.0 and 4.0, such as between 1.0 and 2.0, and in particular between 1.1 and 1.4. Linear alkyl polyglycosides are used, which is to say alkyl polyglycosides in which the polyglycol group is a glucose group and the alkyl group is an n-alkyl group.

Non-ionic surfactants of the amine oxide type, for example N-cocoalkyl-N,N-dimethylamine oxide and N-tallow alkyl-N,N-dihydroxyethylamine oxide, and of the fatty acid alkanolamides may also be suitable. The quantity of these non-ionic surfactants is no more than that of the ethoxylated fatty alcohols, in particular no more than half thereof.

Further suitable surfactants are polyhydroxy fatty acid amides of formula (III),

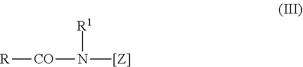

in which RCO denotes an aliphatic acyl group having 6 to 22 carbon atoms, R¹ denotes hydrogen, an alkyl or hydroxyalkyl group having 1 to 4 carbon atoms, and [Z] denotes a linear or branched polyhydroxyalkyl group having 3 to 10 carbon atoms and 3 to 10 hydroxyl groups. The polyhydroxy fatty acid amides are known substances that can usually be obtained by the reductive amination of a reducing sugar with ammonia, an alkylamine or an alkanolamine, and subsequent acylation with a fatty acid, a fatty acid alkyl ester or a fatty acid chloride.

The group of polyhydroxy fatty acid amides also includes compounds of formula (IV),

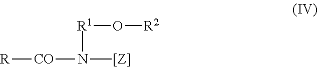

in which R denotes a linear or branched alkyl or alkenyl group having 7 to 12 carbon atoms, R¹ denotes a linear, branched or cyclic alkyl group or an aryl group having 2 to 8 carbon atoms, and R² denotes a linear, branched or cyclic alkyl group or an aryl group or an oxy alkyl group having 1 to 8 carbon atoms, C₁₋₄ alkyl or phenyl groups being used, and [Z] denotes a linear polyhydroxyalkyl group, the alkyl chain of which is substituted with at least two hydroxyl groups, or alkoxylated, such as ethoxylated or propoxylated derivatives of this group. [Z] is obtained by the reductive amination of a reduced sugar, for example glucose, fructose, maltose, lactose, galactose, mannose or xylose. The N-alkoxy- or N-aryloxy-substituted compounds can then be converted, by reaction with fatty acid methyl esters in the presence of an alkoxide as the catalyst, to the desired polyhydroxy fatty acid amides, for example according to the teaching of the international application WO-A-95/07331.

Builder substances are another significant group of washing and cleaning agent ingredients. This substance class is understood to cover both organic and inorganic builder substances. These are compounds which may carry out a carrier function in the agents and also act as a water softening substance in use.

Suitable organic builder substances are, for example, the polycarboxylic acids that can be used in the form of the sodium salts thereof, polycarboxylic acids being understood to mean those carboxylic acids that carry more than one acid function. These include, for example, citric acid, adipic acid, succinic acid, glutaric acid, malic acid, tartaric acid, maleic acid, fumaric acid, saccharic acids, aminocarboxylic acids, nitrilotriacetic acid (NTA), provided that the use thereof is not objectionable for ecological reasons, and mixtures thereof. Non-limiting salts are the salts of polycarboxylic acids such as citric acid, adipic acid, succinic acid, glutaric acid, tartaric acid, saccharic acids, methylglycinediacetic acid, glutaminediacetic acid, and mixtures thereof. The acids can also be used per se. In addition to the builder effect, the acids typically also have the property of being an acidification component and are thus also used, for example in the granules, for setting a lower and milder pH of washing or cleaning agents. Particularly noteworthy here are citric acid, succinic acid, glutaric acid, adipic acid, gluconic acid, methylglycinediacetic acid, glutaminediacetic acid and any mixtures thereof.

Polymeric polycarboxylates are also suitable as builders. These are, for example, the alkali metal salts of polyacrylic acid or polymethacrylic acid, for example those having a relative molecular mass of from 500 to 70,000 g/mol. This substance class has already been described in detail above. The (co)polymeric polycarboxylates may be used either as a powder or as an aqueous solution. The content of (co)polymeric polycarboxylates in the agent is from 0.5 to 20 wt. %, in particular from 3 to 10 wt. %.

To improve water solubility, the polymers can also contain allyl sulfonic acids, such as allyloxybenzene sulfonic acid and methallyl sulfonic acid in EP-B-0 727 448, as a monomer. Biodegradable polymers composed of more than two different monomer units are also possible, for example those that, according to DE-A-43 00 772, contain salts of acrylic acid and of maleic acid, and vinyl alcohol or vinyl alcohol derivatives as monomers or, according to DE-C-42 21 381, salts of acrylic acid and of 2-alkylallylsulfonic acid and sugar derivatives as monomers. Further copolymers are those that are described in the German patent applications DE-A-43 03 320 and DE-A-44 17 734 and comprise acrolein and acrylic acid/acrylic acid salts or acrolein and vinyl acetate as monomers. Polymeric aminodicarboxylic acids, the salts thereof or the precursors thereof should likewise be mentioned as further builders. Polyaspartic acids or the salts and derivatives thereof are possible, of which it is disclosed in the German patent application DE-A-195 40 086 that they also exhibit a bleach-stabilizing effect, in addition to cobuilder properties.

Additional suitable builders are polyacetals, which may be obtained by reacting dialdehydes with polyolcarboxylic acids which have 5 to 7 C atoms and at least 3 hydroxyl groups, for example as described in the European patent application EP-A-0 280 223. Non-limiting polyacetals are obtained from dialdehydes such as glyoxal, glutaraldehyde, terephthalaldehyde and mixtures thereof, and from polyol carboxylic acids such as gluconic acid and/or glucoheptonic acid.

Further suitable organic builders are dextrins, for example oligomers or polymers of carbohydrates, which can be obtained by the partial hydrolysis of starches. The hydrolysis can be carried out according to customary methods, for example acid- or enzyme-catalyzed methods. These dextrins are hydrolysis products having an average molar mass in the range of from 400 to 500,000 g/mol. In this case, a polysaccharide having a dextrose equivalent (DE) in the range of from 0.5 to 40, in particular from 2 to 30, is possible, DE being a customary measure for the reducing effect of a polysaccharide compared with dextrose, which has a DE of 100. It is possible to use both maltodextrins having a DE between 3 and 20 and dried glycose syrups having a DE between 20 and 37, and what are known as yellow dextrins and white dextrins having higher molar masses in the range of from 2,000 to 30,000 g/mol. A dextrin is described in the British patent application 94 19 091. Oxidized derivatives of dextrins of this type are the reaction products thereof with oxidizing agents which are capable of oxidizing at least one alcohol function of the saccharide ring to form a carboxylic acid function. Oxidized dextrins of this kind and methods for the preparation thereof are known, for example, from the European patent applications EP-A-0 232 202, EP-A-0 427 349, EP-A-0 472 042 and EP-A-0 542 496, and the international patent applications WO 92/18542, WO-A-93/08251, WO-A-93/16110, WO-A-94/28030, WO-A-95/07303, WO-A-95/12619 and WO-A-95/20608. An oxidized oligosaccharide according to the German patent application DE-A-196 00 018 is also suitable. A product that is oxidized on C₆ of the saccharide ring can be particularly advantageous.

Oxydisuccinates and other derivatives of disuccinates, such as ethylenediamine disuccinate, are further suitable cobuilders. Ethylenediamine-N,N′-disuccinate (EDDS), the synthesis of which is described in U.S. Pat. No. 3,158,615, for example, is used in the form of the sodium or magnesium salts thereof. Glycerol disuccinates and glycerol trisuccinates, as described for example in the U.S. Pat. Nos. 4,524,009, 4,639,325, in the European patent application EP-A-0 150 930 and in the Japanese patent application JP 93/339896, are also possible in this context. Suitable amounts for use in zeolite-containing and/or silicate-containing formulations are from 3 to 15 wt. %.

Further suitable organic cobuilders are, for example, acetylated hydroxycarboxylic acids or the salts thereof, which optionally can also be present in lactone form and comprise at least 4 carbon atoms and at least one hydroxyl group, as well as no more than two acid groups. Cobuilders of this kind are described, for example, in the international patent application WO-A-95/20029.

A further class of substances having cobuilder properties is that of phosphonates. These include, in particular, hydroxyalkane and aminoalkane phosphonates. Among the hydroxyalkanephosphonates, 1-hydroxyethane-1,1-diphosphonate (HEDP) is of particular importance as a cobuilder. It is used as a sodium salt, the disodium salt reacting neutral and the tetrasodium salt reacting alkaline (pH 9). Possible aminoalkane phosphonates include ethylenediamine tetramethylene phosphonate (EDTMP), diethylentriamine pentamethylene phosphonate (DTPMP) and the higher homologs thereof. They are used in the form of the neutrally reacting sodium salt, for example as the hexasodium salt of EDTMP or as the hepta- and octa-sodium salt of DTPMP. Of the class of phosphonates, HEDP is used as a builder. The aminoalkane phosphonates additionally have a pronounced heavy-metal-binding power. Accordingly, it may be possible, in particular if the agents also include bleach, to use aminoalkane phosphonates, in particular DTPMP, or to use mixtures of the mentioned phosphonates.

Moreover, all compounds that are able to form complexes with alkaline earth ions can be used as cobuilders.

A used inorganic builder is finely crystalline, synthetic and bound-water-containing zeolite. The microcrystalline, synthetic and bound-water-containing zeolite that is used is zeolite A and/or P. Zeolite X and mixtures of A, X and/or P, for example a co-crystallizate from zeolites A and X are also suitable, however. The zeolite can be used as a spray-dried powder or also as an undried, stabilized suspension that is still moist from its preparation process. In the event that the zeolite is used in the form of a suspension, it may contain small amounts of additives of non-ionic surfactants as stabilizers, for example 1 to 3 wt. %, based on zeolite, of ethoxylated C₁₂-C₁₈ fatty alcohols having 2 to 5 ethylene oxide groups, C₁₂-C₁₄ fatty alcohols having 4 to 5 ethylene oxide groups, or ethoxylated isotridecanols. Suitable zeolites have an average particle size of less than 10 μm (volume distribution; measuring method: Coulter counter) and contain 18 to 22 wt. %, and in particular 20 to 22 wt. %, of bound water. In embodiments, zeolites are contained in the premix in amounts of from 10 to 94.5 wt. %, such as in amounts of from 20 to 70 wt. %, in particular 30 to 60 wt. %.

Suitable partial substitutes for zeolites are phyllosilicates of natural and synthetic origin. Phyllosilicates of this kind are known from patent applications DE-A-23 34 899, EP-A-0 026 529 and DE-A-35 26 405, for example. The usability thereof is not limited to a specific composition or structural formula. However, smectites are useful, in particular bentonites. Crystalline, layered sodium silicates of the general formula NaMSiₓO₂ₓ₊₁.yH₂O, in which M denotes sodium or hydrogen, x is a number from 1.9 to 4, and y is a number from 0 to 20, and values for x are 2, 3 or 4, are also suitable for the substitution of zeolites or phosphates. Crystalline phyllosilicates of this kind are described, for example, in European patent application EP-A-0 164 514. Non-limiting crystalline phyllosilicates of the aforementioned formula are those in which M represents sodium and x assumes the values 2 or 3. In particular, both β- and δ-sodium disilicates Na₂Si₂O₅.yH₂O are useful.

The builders also include amorphous sodium silicates with a Na₂O:SiO₂ modulus of 1:2 to 1:3.3, such as of 1:2 to 1:2.8 and in particular of 1:2 to 1:2.6, which exhibit retarded dissolution and have secondary washing properties. The retarded dissolution compared with conventional amorphous sodium silicates may have been caused in a variety of ways, for example by way of surface treatment, compounding, compacting/compression or over-drying. As used herein, the term “amorphous” is also understood to mean “X-ray amorphous.” This means that the silicates do not provide any sharp X-ray reflexes in X-ray diffraction experiments, such as those that are typical of crystalline substances, but at most one or more maxima of the scattered X-rays, which have a width of several degree units of the diffraction angle. However, particularly good builder properties may very well also be achieved when the silicate particles provide washed-out or even sharp diffraction maxima in electron diffraction experiments. This is to be interpreted such that the products comprise microcrystalline regions measuring 10 to several hundred nm, values up to a maximum of 50 nm and in particular up to a maximum of 20 nm being useful. “X-ray amorphous silicates” of this kind, which likewise exhibit retarded dissolution compared with conventional water glasses, are described in the German patent application DE-A-44 00 024, for example. In particular, compressed/compacted amorphous silicates, compounded amorphous silicates and overdried X-ray amorphous silicates are useful, in particular the overdried silicates also occurring as carriers in the granules or being used as carriers in the method.

It is of course also possible to use the generally known phosphates as builders, provided that the use thereof should not be avoided for ecological reasons. Sodium salts of orthophosphates, pyrophosphates and in particular tripolyphosphates are particularly suitable. The content thereof is generally no more than 25 wt. %, such as no more than 20 wt. %, in each case based on the finished agent. In non-limiting embodiments, the agents are phosphate-free, i.e. contain less than 1 wt. % of such phosphates.

In addition to the above-mentioned components, the washing and cleaning agents can additionally include one or more substances from the groups of bleaching agents, bleach activators, enzymes, pH-adjusters, fluorescing agents, dyes, foam inhibitors, silicone oils, anti-redeposition agents, optional brighteners, graying inhibitors, dye transfer inhibitors, corrosion inhibitors, and silver protection agents. Suitable agents are known in the prior art.

This list of washing and cleaning agent ingredients is by no means exhaustive, but merely reflects the most essential typical ingredients of such agents. In particular, the agents may also include organic solvents, if liquid or gel-like preparations are involved. Non-limiting monohydric or polyhydric alcohols having 1 to 4 carbon atoms are used. Non-limiting alcohols in such agents are ethanol, 1,2-propanediol, glycerol, and mixtures of these alcohols. In non-limiting embodiments, agents of this type contain 2 to 12 wt. % of such alcohols.

In principle, the agents may be in different states of aggregation. In a non-limiting embodiment, the washing or cleaning agents are liquid or gel-like agents, in particular liquid washing agents or liquid dishwashing agents or cleaning gels, it being possible in particular for these to also be gel-like cleaning agents for flushing toilets. Such gel-like cleaning agents for flushing toilets are described, for example, in the German patent application DE-A-197 158 72.

Further typical cleaning agents that may be contained in the pro-fragrances are liquid or gel-like cleaners for hard surfaces, in particular those known as all-purpose cleaners, glass cleaners, floor or bathroom cleaners, and special embodiments of such cleaners, which also include acid or alkaline forms of all-purpose cleaners, as well as glass cleaners having what is known as anti-rain action. These liquid cleaning agents can be present either in one or in multiple phases. In a particularly non-limiting embodiment, the cleaners have two different phases.

Cleaner, in the broadest sense, is a designation for, usually surfactant-containing, formulations having a very wide range of use and, dependent thereon, a widely varying composition. The most important market segments are household cleaners, industrial (technical) and institutional cleaners. Based on the pH, a distinction is made between alkaline, neutral and acid cleaners, and according to the form in which the product is offered, a distinction is made between liquid and solid cleaners (including in tablet form). Contrary to dishwashing agents, for example, which can likewise be categorized in the cleaner product group, what are known as cleaners for hard surfaces exhibit an optimal application profile, both in the concentrated state and in a diluted aqueous solution, in conjunction with mechanical energy. Cold cleaners develop the action thereof without an increased temperature. What is decisive for the cleaning action is above all the surfactants and/or alkali carriers, alternatively acids, optionally also solvents such as glycol ethers and lower alcohols. In general, the formulations moreover include builders, and depending on the type of cleaner also bleaching agents, enzymes, microbe-mitigating or disinfecting additives, perfume oils and dyes. Cleaners can also be formulated as microemulsions. The cleaning success, to a large degree, depends on the type of soiling, which also varies widely geographically, and the properties of the surfaces to be cleaned.

The cleaners can include anionic, non-ionic, amphoteric or cationic surfactants as the surfactant component, or surfactant mixtures of one, more or all these surfactant classes. The cleaners contain surfactants in amounts, based on the composition, of from 0.01 to 30 wt. %, such as 0.1 to 20 wt. %, e.g. 1 to 14 wt. %, or 3 to 10 wt. %.

Suitable non-ionic surfactants in such all-purpose cleaners are, for example, C₈-C₁₈ alkyl alcohol polyglycol ethers, alkyl polyglycosides and nitrogen-containing surfactants and mixtures thereof, in particular of the first two. The agents contain non-ionic surfactants in amounts, based on the composition, of from 0 to 30 wt. %, such as 0.1 to 20 wt. %, in particular 0.5 to 14 wt. %, or 1 to 10 wt. %.

C₈₋₁₈ alkyl alcohol polypropylene glycol/polyethylene glycol ethers are known non-ionic surfactants. They can be described by the formula RⁱO—(CH₂CH(CH₃))ₚ(CH₂CH₂O)ₑ—H, in which Rⁱ denotes a linear or branched aliphatic alkyl and/or alkenyl group having 8 to 18 carbon atoms, p denotes 0 or numbers from 1 to 3, and e denotes numbers from 1 to 20. The C₈₋₁₈ alkyl alcohol polyglycol ethers can be obtained by way of addition of propylene oxide and/or ethylene oxide to alkyl alcohols, compared to fatty alcohols. Typical examples are polyglycol ethers in which Rⁱ denotes an alkyl group having 8 to 18 carbon atoms, p denotes 0 to 2, and e denotes numbers from 2 to 7. Non-limiting representatives are, for example, C₁₀-C₁₄ fatty alcohol+1PO+6EO ether (p=1, e=6), and C₁₂-C₁₈ fatty alcohol+7EO ether (p=0, e=7) and mixtures thereof.

It is also possible to use end-capped C₈-C₁₈ alkyl alcohol polyglycol ethers, which is to say compounds in which the free OH group is etherified. The end-capped C₈₋₁₈ alkyl alcohol polyglycol ethers can be obtained according to relevant methods of preparative organic chemistry. In a non-limiting embodiment, C₈₋₁₈ alkyl alcohol polyglycol ethers are reacted in the presence of bases with alkyl halides, in particular butyl or benzyl chloride. Typical examples are mixed ethers, in which Rⁱ denotes a technical fatty alcohol group, such as a C_{12/4} coconut alkyl group, p denotes 0, and e denotes 5 to 10, which are capped with a butyl group.

Furthermore, the alkyl polyglycosides already described above are non-ionic surfactants.

Nitrogen-containing surfactants may be present as further non-ionic surfactants, such as fatty acid polyhydroxyamides, for example glucamides and ethoxylates of alkylamines, vicinal diols and/or carboxylic acid amides that include alkyl groups having 10 to 22 C atoms, such as 12 to 18 C atoms. The degree of ethoxylation of these compounds is generally between 1 and 20, such as between 3 and 10. Ethanolamide derivatives of alkanoic acids having 8 to 22 C atoms, such as 12 to 16 C atoms, are useful. Particularly suitable compounds include lauric acid, myristic acid and palmitic acid monoethanolamides.

Anionic surfactants suitable for all-purpose cleaners are C₈₋₁₈ alkyl sulfates, C₈₋₁₈ alkyl ether sulfates, which is to say the sulfating products of alcohol ethers, and/or C₈₋₁₈ alkylbenzene sulfonates, but also C₈₋₁₈ alkane sulfonates, C₁₋₁₈, α-olefin sulfonates, sulfonated C₈₋₁₈ fatty acids, in particular dodecylbenzene sulfonate, C₈₋₂₂ carboxylic acid amide ether sulfates, sulfosuccinic acid mono- and di-C₁₋₁₂ alkyl esters, C₁₋₁₈, alkyl polyglycol ether carboxylates, C₈₋₁₈ N-acyl taurides, C₈₋₁₈ N-sarcosinates, and C₈₋₁₈ alkyl isethionates, and mixtures thereof. They are used in the form of the alkali metal and alkaline-earth metal salts thereof, in particular sodium, potassium and magnesium salts, and ammonium- and mono-, di-, tri- or tetra-alkyl ammonium salts, and, in the case of the sulfonates, also in the form of the corresponding acid thereof, such as dodecylbenzene sulfonic acid. The agents contain anionic surfactants in amounts, based on the composition, of from 0 to 30 wt. %, such as 0.1 to 20 wt. %, in particular 1 to 14 wt. %, and such as 2 to 10 wt. %.

Due to the foam-controlling properties thereof, the all-purpose cleaners can also include soaps, which is to say alkali or ammonium salts of saturated or unsaturated C₈₋₂₂ fatty acids. The soaps may be used in an amount of up to 5 wt. %, such as from 0.1 to 2 wt. %.

Suitable amphoteric surfactants are, for example, betaines of formula (Rⁱ)(Rⁱⁱ)(R^{iv})N⁺CH₂COO⁻, in which Rⁱⁱ denotes an alkyl group, which is optionally interrupted by heteroatoms or heteroatom groups, having 8 to 25, such as 10 to 21, carbon atoms, and Rⁱⁱ and R^{iv} denote identical or different alkyl groups having 1 to 3 carbon atoms, in particular C₁₀₋₁₈ alkyl dimethyl carboxymethyl betaine and C₁₁₋₁₇ alkyl amido propyl dimethyl carboxymethyl betaine. The agents contain amphoteric surfactants in amounts, based on the composition, of from 0 to 15 wt. %, such as 0.01 to 10 wt. %, and in particular 0.1 to 5 wt. %.

Suitable cationic surfactants are, inter alia, the quaternary ammonium compounds of formula (R^{v})(R^{vi})(R^{vii})(R^{viii})N⁺X⁻, in which R^{v} to R^{vii} denote four identical or different, and in particular two long-chain and two short-chain, alkyl groups, and X⁻ denotes an anion, in particular a halide ion, for example didecyl dimethyl ammonium chloride, alkyl benzyl didecyl ammonium chloride and mixtures thereof. The agents contain cationic surfactants in amounts, based on the composition, of from 0 to 10 wt. %, such as 0.01 to 5 wt. %, and in particular 0.1 to 3 wt. %.

In a non-limiting embodiment, the cleaners contain anionic and non-ionic surfactants adjacent to one another, such as C₈₋₁₈ alkylbenzene sulfonates, C₈₋₁₈ alkyl sulfates and/or C₈₋₁₈ alkyl ethersulfates adjacent to C₈₋₁₈ alkyl alcohol polyglycol ethers and/or alkyl polyglycosides, in particular C₈₋₁₈ alkylbenzene sulfonates adjacent to C₈₋₁₈ alkyl alcohol polyglycol ethers.

The cleaners can moreover contain builders. Suitable builders are, for example, alkali metal gluconates, citrates, nitrilotriacetates, carbonates and bicarbonates, in particular sodium gluconate, citrate and nitrilotriacetate, and sodium and potassium carbonate and bicarbonate, and alkali metal and alkaline-earth metal hydroxides, in particular sodium and potassium hydroxide, ammonia and amines, in particular monoethanolamine and triethanolamine, and mixtures thereof. The salts of glutaric acid, succinic acid, adipic acid, tartaric acid and benzene hexacarboxylic acid as well as phosphonates and phosphates are included in this category. The agents contain builders in amounts, based on the composition, of from 0 to 20 wt. %, such as 0.01 to 12 wt. %, in particular 0.1 to 8 wt. %, and such as 0.3 to 5 wt. %, the amount of sodium hexametaphospate, excluding the agents used, being limited to 0 to 5 wt. %, however. Serving as electrolytes, the builder salts are auxiliary phase separation agents at the same time.

In addition to the mentioned components, the cleaners may contain further auxiliary agents and additives as are common in such agents. These include in particular polymers, soil release active ingredients, solvents (such as ethanol, isopropanol, glycol ether), solubilizers, hydrotropic substances (such as cumene sulfonate, octyl sulfate, butyl glucoside, butyl glycol), dry-cleaning detergents, viscosity regulators (e.g. synthetic polymers such as polysaccharides, polyacrylates, naturally occurring polymers and the derivatives thereof, such as xanthan gum, other polysaccharides and/or gelatin), pH regulators (such as citric acid, alkanolamines or NaOH), disinfectants, antistatic agents, preservatives, bleaching systems, enzymes, dyes, and opacifying agents or skin protection agents, as they are described in EP-A-0 522 506. The amount of additives of this type in the cleaning agent is usually no greater than 12 wt. %. The lower use limit depends on the additive type and may for example be as low as 0.001 wt. % or less for dyes. The amount of auxiliaries is between 0.01 and 7 wt. %, in particular 0.1 and 4 wt. %.

The pH of the all-purpose cleaners can be varied across a wide range; however, a range of from 2.5 to 12, and in particular 5 to 10.5 is useful. The pH shall be understood to mean the pH of the agent in the form of the temporary emulsion.

Such all-purpose cleaner formulations can be modified for any purpose. Glass cleaners form a particular embodiment. In cleaners of this kind it is essential that stains or outlines remain. In particular, it is a problem that, after cleaning, water condenses on these surfaces and results in what is known as the fogging effect. It is likewise undesirable when what are known as rain stains remain on glass panes exposed to rain. This effect is known as rain effect, or anti-rain effect. These effects can be prevented by suitable additives in glass cleaners.

In another embodiment, the agents are powdery or granular agents. The agents can have any bulk density. The spectrum of possible bulk densities ranges from low bulk densities of less than 600 g/l, such as 300 g/l, through the range of average bulk densities of from 600 to 750 g/l, to the range of high bulk densities of at least 750 g/l.

Any methods known from the prior art are suitable for preparing such agents.

Cosmetic agents for hair or skin treatment may contain the pro-fragrances described herein in the amounts already described above in combination with the other agents. In a non-limiting embodiment, the cosmetic agents are aqueous preparations that contain surface-active ingredients and that are suitable in particular for treating keratin fibers, in particular human hair, or for treating skin.

The mentioned hair treatment agents are in particular agents for treating human scalp hair. The most common agents of this category can be divided into hair washing agents, hair care agents, hair setting agents and hair styling agents, as well as hair dyes and hair removal agents. The agents containing surface-active ingredients include in particular hair washing agents and hair care agents. These aqueous preparations are typically present in a liquid to pasty form.

Fatty alcohol polyglycol ether sulfates (ether sulfates, alkyl ether sulfates), at times in combination with other usually anionic surfactants, are used predominantly for the most important group of ingredients, this being the surface-active ingredients or substances that provide washing action. In addition to good cleaning power and insensitivity to water hardness, shampoo surfactants should be tolerated by the skin and mucous membranes. In accordance with statutory provisions, they must be easily biodegradable. In addition to alkyl ether sulfates, agents can additionally contain further surfactants such as alkyl sulfates, alkyl ether carboxylates, having degrees of ethoxylation of from 4 to 10, and surfactant protein/fatty acid condensates.

Hair shampoos contain perfume oils to produce an appealing fragrance note. In this case, the shampoos may contain only the pro-fragrances, but it is also useful if the hair shampoos contain not only these, but also other fragrances. Any conventional fragrances authorized for use in hair shampoos may be used in this case.

The goal of hair care agents is to preserve the natural state of newly regrown hair for as long as possible, and to restore same if damaged. Features that characterize this natural state are a silky sheen, low porosity, an elastic, yet soft volume, and a pleasantly smooth feel. An important prerequisite for this is a clean, not overly oily scalp that is free of dandruff. Today, a plurality of different products are covered by hair care agents, the most important representatives of which are referred to as pre-treatment agents, hair tonics, hair styling aids, hair conditioners and deep conditioning products.

The aqueous preparations for treating skin are in particular preparations for human skin care. This care begins with cleansing, for which primarily soaps are used. In this regard, a distinction is made between solid soap, usually in bars, and liquid soap. Accordingly, in a non-limiting embodiment the cosmetic agents are present as shaped bodies that contain surface-active ingredients. In a non-limiting embodiment, the most important ingredients of such shaped bodies are the alkali salts of fatty acids of natural oils and fats, having chains of 12-18 C atoms. Since lauric acid soaps lather particularly well, coconut and palm kernel oils rich in lauric acid are useful raw materials for fine soap production. The Na salts of fatty acid mixtures are solid; the K salts are soft-pasty. For saponification, the diluted caustic soda or caustic potash is added to the fat raw materials at a stoichiometric ratio so that an excess of lye of no more than 0.05% is present in the finished soap. In many instances, soaps today are no longer produced directly from the fats, but from the fatty acids obtained by way of lipolysis. Customary soap additives are fatty acids, fatty alcohols, lanolin, lecithin, vegetable oils, partial glycerides, inter alia fat-like substances for lipid replenishment of the cleansed skin, antioxidants such as ascorbyl palmitate or tocopherol for preventing auto-oxidation of the soap (rancidity), complexing agents such as nitrilotriacetate for binding heavy metal traces that could catalyze the auto-oxidative spoilage, perfume oils for achieving the desired fragrance notes, dyes for coloring the bars of soap, and optionally special additives.

Liquid soaps are based on both K salts of natural fatty acids and on synthetic anionic surfactants. In aqueous solution, they contain fewer substances that provide washing action than solid soaps, and include the customary additives, optionally including viscosity-regulating components, and pearlescing additives. Due to the convenient and hygienic application from dispensers, they are used in public lavatories and the like. Body washes for particularly sensitive skin are based on synthetic surfactants having a mild action, to which skin care substances are added and which are set to a neutral or slightly acidic pH (pH 5.5).

For cleansing primarily facial skin, a number of additional preparations are available, such as facial toners, cleansing-lotions, -milks, -creams and -pastes; some face packs are used for cleansing, but they generally refresh and nourish the facial skin. Facial toners are typically aqueous-alcoholic solutions having a low surfactant content and containing further skin care substances. Cleansing-lotions, -milks, -creams and -pastes are typically based on O/W emulsions that have a relatively low fatty component content and contain cleansing and nourishing additives. What are known as scruffing and peeling preparations contain substances that have a mild keratolytic effect to remove the upper dead skin-horn layers; in some instances these preparations also contain an added abrasively acting powder. Almond bran, which has been used as a mild cleansing care agent for quite some time, is frequently still a component of such preparations today. Agents for the cleansing treatment of blemished skin also contain antibacterial and anti-inflammatory substances, since the accumulation of sebaceous material in comedones (blackheads) represents a breeding ground for bacterial infections and tends cause inflammation. The wide range of different skin cleansing products offered varies in terms of the composition and content of different active ingredients depending on different skin types and specific treatment purposes.

Further cosmetic agents are agents for influencing body odor. This refers in particular to deodorizing agents. Such deodorants are able to mask, remove or destroy odors. Unpleasant body odors arise from the bacterial decomposition of sweat, in particular in the warm and moist axilla regions, where microorganisms encounter good living conditions. As a result, antimicrobial substances are the most important ingredients of deodorants. In particular, antimicrobial substances that have a substantially selective effectiveness with respect to bacteria responsible for body odor are useful. Non-limiting active ingredients, however, have only a bacteriostatic effect and by no means completely destroy the bacterial flora. Antimicrobial agents include in general all suitable preservatives that specifically work against gram-positive bacteria. These are, for example, Irgasan DP 300 (trichlosan, 2,4,4′-trichloro-2′-hydroxydiphenyl ether), chlorhexidine (1,1′-hexamethylenebis (5-(4′-chlorophenyl)-biguanide) and 3,4,4-trichlorocarbanilide. In principle, quaternary ammonium compounds are likewise suitable. Due to the high antimicrobial effectiveness, all these substances are used only in low concentrations of approximately 0.1 to 0.3 wt. %. Moreover, numerous odorants also exhibit antimicrobial properties. Accordingly, such odorants having antimicrobial properties are used in deodorants. In particular, farnesol and phenoxyethanol shall be mentioned here. The deodorants may contain odorants which are themselves bacteriostatically acting. The odorants can be contained again in the form of the pro-fragrances. However, it is also possible that precisely these antibacterially active odorants are not used in the form of pro-fragrances and then used in mixtures with other fragrances which are present as pro-fragrances. A further group of essential ingredients of deodorants are enzyme inhibitors, which inhibit the enzymatic decomposition of sweat, such as triethyl citrate or zinc glycinate, for example. Essential ingredients of deodorants are furthermore also antioxidants, which are intended to prevent oxidation of sweat components.

In a further likewise embodiment, the cosmetic agent is a hair setting agent that contains polymers for setting. At least one polyurethane may be contained among the polymers.

Finally, air care products, for example in the form of sprays, and insect repellents, which in addition to the pro-fragrances described herein may contain the typical and known ingredients of such agents.

In principle, all embodiments disclosed in connection with the pro-fragrances and the agents are also applicable to the described methods and uses, and vice versa. It goes without saying, for example, that all special pro-fragrances described herein can be used in the described agents and methods and can be used as described herein.

### EXAMPLES

### Example 1: Pro-Fragrance Dimer with Benzylacetone

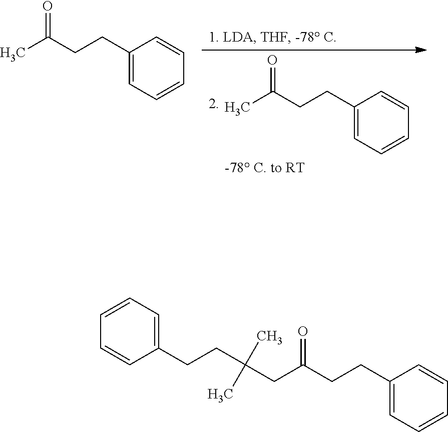 [SMILES: CC(=O)CCc1ccccc1 ; CC(=O)CCc1ccccc1 ; CC(C)(CCc1ccccc1)CC(=O)CCc1ccccc1]

The reaction flask was heated several times under high vacuum (approximately 0.1 mbar) using a gas burner and aerated with argon, diisopropylamine (4.07 g, 40.3 mmol) in 5 ml THF was placed, cooled to approximately −78° C. and n-butyllithium (16.8 ml, 42 mmol, 2.5 M in hexane) was slowly added over 15 min. Subsequently, benzylacetone (5.2 g, 35 mmol) in 5 ml THF was added. The yellow reaction solution was stirred for 30 minutes at −78° C. After addition of benzylacetone (5.2 g, 35 mmol) in 5 ml THF, this was stirred for a further 90 min at −78° C. and heated to room temperature for 3 hours. The reaction was terminated by the addition of NaHCO₃ solution and extraction with diethyl ether. The combined organic phases were dried over MgSO₄, filtered and concentrated to dryness. Column chromatographic purification (pentane+1% triethylamine; ethyl acetate) gave the desired product as a yellow viscous oil (1.98 g, 6.68 mmol, 19%).

R_{f}(PE:EE, 10:1)=0.05. IR (film): \tilde{v}=3482 cm⁻¹, 3081, 3062, 3027, 2967, 2932, 2862, 1698, 1603, 1497, 1454, 1373, 1062, 1030, 921, 743, 697, 492. ¹H-NMR (CDCl₃, 400 MHz): δ=1.25 (s, 3H), 1.70-1.85 (m, 2H), 2.54 (d, J=12.9 Hz, 1H), 2.61 (d, J=13.0 Hz, 1H), 2.57-2.77 (m, 4H), 2.86 (t, J=7.1 Hz, 2H), 3.84 (s, 1H) 7.15-7.20 (m, 6H), 7.23-7.30 (m, 4H). ¹³C-NMR (CDCl₃, 101 MHz): δ=25.7 (q), 29.3 (t), 30.2 (t), 43.7 (t), 45.9 (t), 51.8 (t), 71.5 (s), 125.7 (d), 126.2 (d), 128.2 (d, 2C), 128.3 (d, 2C), 128.3 (d, 2C), 128.5 (d, 2C), 140.5 (s), 142.2 (s), 212.1 (s).

### Example 2: Pro-Fragrance Dimer with 2-Undecanone

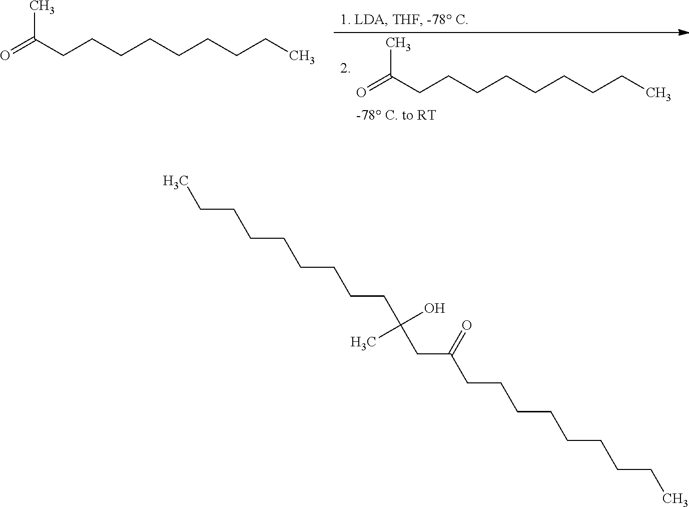 [SMILES: CCCCCCCCCC(C)=O ; CCCCCCCCCC(C)=O ; CCCCCCCCCC(=O)CC(C)(O)CCCCCCCCC]

The reaction flask was heated several times under high vacuum (approximately 0.1 mbar) using a gas burner and aerated with argon, diisopropylamine (3.33 g, 99.0 mmol) in 15 ml THF was placed, cooled to approximately −78° C. and n-butyllithium (13.2 ml, 99.0 mmol, 2.5 M in hexane) was slowly added over 15 min. Subsequently, 2-undecanone (15.3 g, 90 mmol) in 15 ml THE was added. The yellow reaction solution was stirred for 30 minutes at −78° C. After addition of 2-undecanone (15.3 g, 90 mmol) in 15 ml THF, this was stirred for a further 90 min at −78° C. and heated to room temperature for 3 hours. The reaction was terminated by the addition of NaHCO₃ solution and extraction with diethyl ether. The combined organic phases were dried over MgSO₄, filtered and concentrated to dryness. The crude product was purified by column chromatography (pentane+1% triethylamine, ethyl acetate), excess 2-undecanone was distilled off in a vacuum (0.1 mbar, 60° C. ramp 120° C. oil bath) and the product obtained crystallized at 8° C. to colorless solid (11.09 g, gave the desired product as a yellow viscous oil (11.09 g, 32.6 mmol, 36%).

R_{f}(PE:EE, 10:1)=0.44. IR (film): \tilde{v}=3504 cm¹, 2923, 2854, 1701, 1466, 1375, 1310, 1141, 1076, 931, 988, 721, 526. ¹H-NMR (CDCl₃, 400 MHz): δ=0.88 (t, J=6.8 Hz, 6H), 1.19 (s, 3H), 1.22-1.36 (m, 26H), 1.42-1.51 (m, 2H), 1.52-1.62 (m, 2H), 2.41 (t, J=7.6 Hz, 2H), 2.53 (d, J=17.1 Hz, 1H), 2.60 (d, J=17.2 Hz, 1H), 3.30-4.20 (bs, 1H). ¹³C-NMR (CDCl₃, 101 MHz): δ=14.2 (q, 2C), 22.8 (t, 2C), 23.6 (t), 24.1 (t), 26.9 (q), 29.2 (t), 29.4 (t, 2C), 29.5 (t, 2C), 29.7 (t, 2C), 30.2 (t), 32.0 (t, 2C), 42.4 (t), 44.8 (t), 51.4 (t), 71.8 (s), 213.7 (s). MS (EI, 70 eV): m/z (%): 397 (13) [M+TMS-Me]⁺, 285 (100), 243 (45), 155 (37), 73 (24), 43 (15).

### Example 3: TGA/IR Test of the 2-Undecanone Dimer from Example 2

Using TGA, the sample shows a complete linear weight reduction under nitrogen between about 100° C. and 300° C. The weight residues of the sample at different temperatures are given in Table 1. At the beginning of the measurement, the sample shows bands of water (H₂O) and carbon dioxide (CO₂) in the spectrum of the gas stream.

Furthermore, the gas phase spectrum has bands of 2-undecanone. This shows that at higher temperatures, a retro-aldol reaction takes place, which releases the desired molecules again.

TABLE 1
Weight residue at . . . ° C. in wt. % after approx . . . min.

| RT | 100° C. | 200° C. | 300° C. | 400° C. |
|---|---|---|---|---|
| 0 min | 7 min | 17 min | 27 min | 37 min |
| 100.0 | 99.8 | 83.2 | 0.1 | 0.0 |
| (±0.0) | (±0.0) | (±1.3) | (±0.1) | (±0.1) |

1. A pro-fragrance comprising the formula (I): 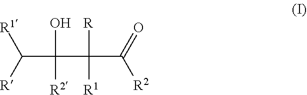
wherein:
R, R′, R¹, R^{1′}, R² and R^{2′} are independently selected from H, straight-chain or branched, saturated or unsaturated, substituted or unsubstituted hydrocarbon groups having from 1 to 20 carbon atoms and optionally up to 6 heteroatoms; cycloalkyl or cycloalkenyl having up to 20 carbon atoms; heterocycloalkyl or heterocycloalkenyl having up to 20 carbon atoms and from 1 to 6 heteroatoms selected from O, S, N, and combinations thereof; or
R² and R¹ or R and R² or R^{2′} and R^{1′} or R^{2′} and R′ combine to form a cyclic group selected from substituted or unsubstituted aryl having up to 20 carbon atoms, substituted or unsubstituted heteroaryl having up to 20 carbon atoms and 1 to 6 heteroatoms selected from O, S, N, and combinations thereof; substituted or unsubstituted cycloalkyl or cycloalkenyl having up to 20 carbon atoms; substituted or unsubstituted heterocycloalkyl or heterocycloalkenyl having up to 20 carbon atoms and 1 to 6 heteroatoms selected from O, S, and N, and combinations thereof;
wherein at least one of R, R¹, and R² and at least one of R′, R^{1′} and R^{2′} is not H; wherein the group —(CRR¹)—C(O)R² is derived from a fragrance ketone of formula R²—C(O)—CHRR¹; and wherein the group —COHR^{2′}—CR′R^{1′} is derived from a fragrance ketone of formula R^{2′}—C(O)—CHR′R^{1′}.

2. The pro-fragrance according to claim 1, wherein R² and/or R^{2′} is a straight-chain or branched, saturated or unsaturated, substituted or unsubstituted hydrocarbon group having 1 to 20 carbon atoms and optionally up to 6 heteroatoms.

3. The pro-fragrance according to claim 1,
wherein either R¹ or R is H, R^{1′} or R′ is H, or combinations thereof, and wherein the other group is a straight-chain or branched, saturated or unsaturated, substituted or unsubstituted hydrocarbon group having 1 to 20 carbon atoms and optionally up to 6 heteroatoms; or
wherein R¹ and R are H, R^{1′} and R′ are H, or both.

4. The pro-fragrance according to claim 1, wherein R¹ and R are H and R² is a substituted or unsubstituted linear alkyl group having up to 12 carbon atoms.

5. The pro-fragrance according to claim 1, wherein:
R═R′;
R¹═R^{1′};
R²═R^{2′}, or
combinations thereof.

6. The pro-fragrance according to claim 1, wherein the fragrance ketone of formula R²—C(O)—CHRR¹ and/or R^{2′}—C(O)—CHR′R^{1′} is selected from the group consisting of the following fragrance ketones: 2-undecanone (methyl nonyl ketone), methyl beta naphthyl ketone, musk indanone (1,2,3,5,6,7-hexahydro-1,1,2,3,3-pentamethyl-4H-inden-4-one), tonalide (6-acetyl-1,1,2,4,4,7-hexamethyltetralin), alpha-damascone, beta-damascone, delta-damascone, iso-damascone, damascenone, methyldihydrojasmonate, menthone, carvone, camphor, koavone (3,4,5,6,6-pentamethylhept-3-en-2-one), fenchone, alpha-ionone, beta-ionone, gamma-methyl-ionone, fleuramone (2-heptylcyclopentanone), dihydrojasmone, cis-jasmone, 1-(1,2,3,4,5,6,7,8-octahydro-2,3,8,8-tetramethyl-2-naphthalenyl)-ethan-1-one and isomers thereof, methyl cedrenyl ketone, acetophenone, methyl acetophenone, para-methoxy acetophenone, methyl beta-naphthyl ketone, benzyl acetone, benzophenone, para-hydroxyphenyl butanone, celery ketone (3-methyl-5-propyl-2-cyclohexenone), 6-isopropyl decahydro-2-naphtone, dimethyloctenone, Frescomenthe (2-butan-2-yl-cyclohexan-1-one), 4-(1-ethoxyvinyl)-3,3,5,5-tetramethylcyclohexanone, methyl heptenone, 2-(2-(4-methyl-3-cyclohexen-1-yl)propyl)cyclopentanone, 1-(p-menthen-6(2)yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)-indanone, 4-damascol, dulcinyl (4-(1,3-benzodioxol-5-yl)butan-2-one), hexalone (1-(2,6,6-trimethyl-2-cyclohexen-1-yl)-1,6-heptadien-3-one), Isocyclemone E (2-acetonaphthone-1,2,3,4,5,6,7,8-octahydro-2,3,8,8-tetramethyl), methyl nonyl ketone, methyl cyclocitrone, methyl lavender ketone, orivone (4-tert-amylcyclohexanone), 4-tert-butylcyclohexanone, delphone (2-pentyl-cyclopentanone), muscone (CAS 541-91-3), neobutenone (1-(5,5-dimethyl-1-cyclohexenyl)pent-4-en-1-one), plicatone (CAS 41724-19-0), veloutone (2,2,5-trimethyl-5-pentylcyclopentan-1-one),2,4,4,7-tetramethyl-oct-6-en-3-one, tetrameran (6,10-dimethylundecen-2-one), or combinations thereof.

7. (canceled)

8. A composition comprising:
an agent selected from the group comprising washing agent,
a cleaning agent, a cosmetic agent, an air care product,
an insect repellent, or combinations thereof; and
the pro-fragrance according to claim 1.

9. The composition according to claim 8, wherein the agent is
(a) a liquid or gel-like agent;
(b) a powdery or granular agent;
(c) an agent in the form of shaped bodies;
(d) a cosmetic hair or skin treatment agent;
(e) or combinations thereof.

10. The composition according to claim 8, wherein the pro-franqrance is present in an ranging from 0.001 to 5 wt. % in each case based on the agent.

11. The composition of claim 8, wherein formula (I) of the profragrance comprises R² and/or R^{2′} being a straight-chain or branched, saturated or unsaturated, substituted or unsubstituted hydrocarbon group having 1 to 20 carbon atoms and optionally up to 6 heteroatoms.

12. The composition according to claim 8, wherein formula (I) of the profragrace comprises either R¹ or R being H, R^{1′} or R′ being H, or combinations thereof; and wherein the other group is a straight-chain or branched, saturated or unsaturated, substituted or unsubstituted hydrocarbon group having 1 to 20 carbon atoms and optionally up to 6 heteroatoms; or
wherein R¹ and R are H, R^{1′} and R′ are H, or both.

13. The composition according to claim 8, wherein formula (I) of the profragrace comprises R¹ and R being H and R² is a substituted or unsubstituted linear alkyl group having up to 12 carbon atoms.

14. The composition according to claim 8, wherein formula (I) of the profragrace comprises:
R═R′;
R¹═R^{1′};
R²═R^{2′}, or
combinations thereof.

15. The composition according to claim 8, wherein the fragrance ketone of formula R²—C(O)—CHRR¹ and/or R^{2′}—C(O)—CHR′R^{1′} is selected from the group consisting of the following fragrance ketones: 2-undecanone (methyl nonyl ketone), methyl beta naphthyl ketone, musk indanone (1,2,3,5,6,7-hexahydro-1,1,2,3,3-pentamethyl-4H-inden-4-one), tonalide (6-acetyl-1,1,2,4,4,7-hexamethyltetralin), alpha-damascone, beta-damascone, delta-damascone, iso-damascone, damascenone, methyldihydrojasmonate, menthone, carvone, camphor, koavone (3,4,5,6,6-pentamethylhept-3-en-2-one), fenchone, alpha-ionone, beta-ionone, gamma-methyl-ionone, fleuramone (2-heptylcyclopentanone), dihydrojasmone, cis-jasmone, 1-(1,2,3,4,5,6,7,8-octahydro-2,3,8,8-tetramethyl-2-naphthalenyl)-ethan-1-one and isomers thereof, methyl cedrenyl ketone, acetophenone, methyl acetophenone, para-methoxy acetophenone, methyl beta-naphthyl ketone, benzyl acetone, benzophenone, para-hydroxyphenyl butanone, celery ketone (3-methyl-5-propyl-2-cyclohexenone), 6-isopropyl decahydro-2-naphtone, dimethyloctenone, Frescomenthe (2-butan-2-yl-cyclohexan-1-one), 4-(1-ethoxyvinyl)-3,3,5,5-tetramethylcyclohexanone, methyl heptenone, 2-(2-(4-methyl-3-cyclohexen-1-yl)propyl)cyclopentanone, 1-(p-menthen-6(2)yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4(5H)-indanone, 4-damascol, dulcinyl (4-(1,3-benzodioxol-5-yl)butan-2-one), hexalone (1-(2,6,6-trimethyl-2-cyclohexen-1-yl)-1,6-heptadien-3-one), Isocyclemone E (2-acetonaphthone-1,2,3,4,5,6,7,8-octahydro-2,3,8,8-tetramethyl), methyl nonyl ketone, methyl cyclocitrone, methyl lavender ketone, orivone (4-tert-amylcyclohexanone), 4-tert-butylcyclohexanone, delphone (2-pentyl-cyclopentanone), muscone (CAS 541-91-3), neobutenone (1-(5,5-dimethyl-1-cyclohexenyl)pent-4-en-1-one), plicatone (CAS 41724-19-0), veloutone (2,2,5-trimethyl-5-pentylcyclopentan-1-one), 2,4,4,7-tetramethyl-oct-6-en-3-one, tetrameran (6,10-dimethylundecen-2-one), or combinations thereof.

16. The composition according to claim 8, wherein formula (I) of the profragrance comprises R² and/or R^{2′} having a linear or branched, substituted or unsubstituted, alkyl, alkenyl or alkynyl group having up to 20 carbon atoms.

17. The composition according to claim 8, wherein formula (I) of the profragrance comprises R² and/or R^{2′} comprising methyl, ethyl, propyl, butyl, pentyl, hexyl, heptyl, octyl, nonyl, decyl, or combinations thereof.

18. The composition of claim 8, wherein formula (I) of the profragrance comprises R² being a substituted linear alkyl group having up to 12 carbon atoms.
